(19) United States
(12) Patent Application Publication
(10) Pub. No.: US 2010/0263151 A1
(43) Pub. Date: Oct. 21, 2010

(54) Myriad Pad

(76) Inventor: Roberta B. Koehl, Hidden Hills, CA (US)

Correspondence Address: Roberta Koehl, 23747 Oakfield Road, Hidden Hills, CA, 91302, US

(21) Appl. No.: 12/427,557

(22) Filed: Apr. 21, 2009

(51) Int. Cl. A61B 17/50; A45D 44/00

(52) U.S. Cl. 15/208; 606/131

(57) ABSTRACT

A unique device that has two sides, one gauze, one cotton, to allow the cleaning and exfoliating of skin. The device can clean makeup, exfoliate, clean wounds, while changing surfaces, remove makeup around the eyes, face and perform all functions normally serviced by a single sided pad or applicator with a stronger secondary surface that increases the number of uses, and makes the item in issue long lasting for comparable price. The cotton is absorbent, the gauze side strong enough to remove all dirt without deteriorating, and do so faster and more efficiently than cotton alone. It is essentially a potentially two-step device, the delicate side for usage first, followed by the stronger gauze covering as deemed necessary. There is no comparable prior or present art.

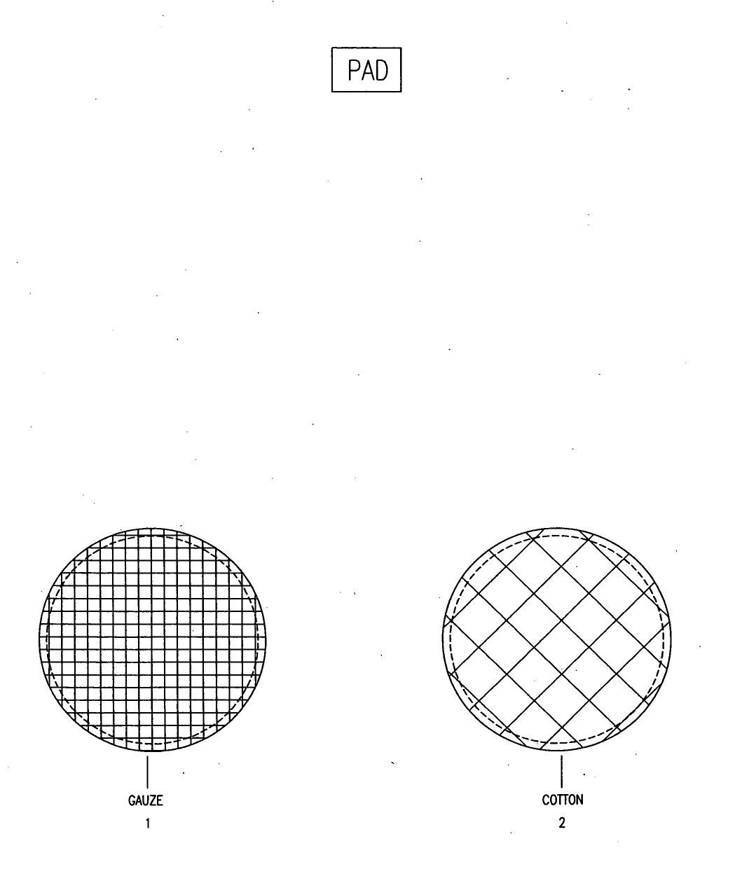

### BACKGROUND OF THE INVENTION

1. The invention itself

One presents a two-sided flat pad applicator, with gauze on one side, and cotton on the other. The two different sides serve two different functions. The cotton is soft, and can deal with delicate areas, as any normal cotton applicator or swab. But the gauze side can deal with heavier problems, stubborn makeup, heavy dirt, etc., where cotton has no real value due to its softness and lack of durability. The device is marginally more expensive than that on the market, but will be competitive. It also has numerous uses not found in the pad with just cotton on the surfaces. It has innumerable uses from personal to medical, per example, the gauze side is strong enough to remove excess makeup and toner, while exfoliating skin. The multiple uses and applications, let alone the differences in the sides and its construction, make the name inevitable. Hence, Myriad Pad.

2. Description of the Prior Art

There is no prior art comparable to the proposed inventions here. The present cotton pad is not competitive. Said pad is at best, a double-sided cotton pad, and has no gauze or stronger surface. It is roughly similar in size, but cannot serve the functions of the “Myriad Pad.” The basis for the Myriad Pad is the lack of usefulness of the present cotton pads, their weakness, and their lack of durability. There is no real competition here and no generic similarities. There is no competition extant. There is no dual purpose item or product on the market at this time.

### SUMMARY OF THE INVENTION

The multiple objectives of the invention is best described by the abstract that follows. They are accompanied by drawings that detail the construction of the pad, and show the actual product itself.

The objects or objectives of the invention are multiple

1. To create a dual purpose cleaning utensil that can both clean and exfoliate delicate areas and harsher surfaces, without the need for additional chemicals or devices, presently unknown on the market;

2. A device that is competitive in cost with the present market, but is more durable, has additional features, is safe to use, and is biodegradable;

3. Create a device that has more uses than merely the cosmetic, and has a durability that allows for the purchase of fewer items, with significantly more use being gained from the items purchased. The environment and the consumer are benefited.

### BRIEF DESCRIPTION OF THE DRAWINGS

The view of the drawing enclosed are self-explanatory, and connection to the physical structure of the device.

### DESCRIPTION OF THE PREFERRED EMBODIMENTS

There are obvious nuances here. One can't be certain of all the uses of this device at this time. No one has ever produced a two-sided invention with multiple surfaces. The supple and absorbent cotton (2) and the rougher exfoliant gauze (1) give this one pad many uses. It may have uses in industries beyond cosmetics, and other facets presently unknown, due to the gauze side. But the application here is cosmetic and bodily cleansing. Industry usage may vary, and those uses will become evident after the product is in service.

What is claimed is:

1. A unique two-sided gauze and cotton device, with one side being cotton, and the other side being gauze, in a disposable format for use on the body and around the face. The device in issue has no comparable prior art; all prior art is two-sided in cotton alone.

2. A unique device, by which an end user can have a soft permeable surface and a rougher more exfoliant surface to handle different bodily needs. The soft cotton side can clean gentle surfaces and the gauze side of the tip can deal safely with dirt, makeup, and skin conditions where a stronger surface is needed and can be safely used.

3. A method of cosmetic treatment that allows more than one usage of a cleaning pad, predicated on the duality of the surfaces. All other present art is limited by cotton coverings, and cannot service the more difficult functions, such as removal of dirt, and stubborn makeup.

4. The invention cleans the skin surface, can exfoliate the same, can clean a wound, and remove nail polish and makeup. Both sides are absorbent, and the gauze side can condition the skin in ways cotton cannot. In addition, the gauze side removes polish and makeup faster and more efficiently than cotton.
